(19) United States
(12) Patent Application Publication
(10) Pub. No.: US 2021/0233317 A1
(43) Pub. Date: Jul. 29, 2021

(54) APPARATUS AND METHOD OF CLINICAL TRIAL FOR VR SICKNESS PREDICTION BASED ON CLOUD

(71) Applicant: ELECTRONICS AND TELECOMMUNICATIONS RESEARCH INSTITUTE, Daejeon (KR)

(72) Inventors: Wook-Ho SON, Daejeon (KR); Hee-Seok OH, Seoul (KR); Beom-Ryeol LEE, Daejeon (KR); Yong-Ho LEE, Daejeon (KR)

(21) Appl. No.: 17/155,267

(22) Filed: Jan. 22, 2021

(30) Foreign Application Priority Data
Jan. 28, 2020 (KR) 10-2020-0009880
Apr. 10, 2020 (KR) 10-2020-0043999

(51) Int. Cl. G06T 19/00; G06F 3/01; G06N 20/00

(52) U.S. Cl. CPC G06T 19/006; G06F 3/015; G06N 20/00; G06F 3/013; G06F 3/012

(57) ABSTRACT

Disclosed herein are an apparatus and method for a clinical trial for predicting the degree of VR sickness based on a cloud. The apparatus for a clinical trial for predicting the degree of VR sickness includes one or more processors and executable memory for storing at least one program executed by the one or more processors. The at least one program provides VR content to a user, extracts clinical data for predicting the degree of motion sickness of each user, and transmits the clinical data to a cloud server.

### CROSS REFERENCE TO RELATED APPLICATIONS

This application claims the benefit of Korean Patent Application No. 10-2020-0009880, filed Jan. 28, 2020, and No. 10-2020-0043999, filed Apr. 10, 2020, which are hereby incorporated by reference in their entireties into this application.

### BACKGROUND OF THE INVENTION

### 1. Technical Field

The present invention relates generally to an apparatus and method for a clinical trial for predicting the degree of VR sickness based on a cloud, and more particularly to technology for predicting the degree of VR sickness occurring when viewing VR content using a Head-Mounted Display (HMD), predicting the degree of motion sickness due to various types of VR image content online or offline, and showing the predicted degree to a user.

### 2. Description of Related Art

Unless otherwise indicated herein, the materials described in this section are not the prior art with regard to the claims in this application, and are not admitted to be prior art by inclusion in this section.

In the case of conventional VR sickness prediction, VR sickness evaluation is conducted for respective individuals using an expensive device installed at a specific location, clinical trial data acquired through the VR sickness evaluation is accumulated, and the degree of VR sickness is quantitatively predicted using a machine-learning method.

However, in order to conduct VR sickness evaluation for each individual, it is required to use an expensive device placed at a specific test location, and it takes at least an hour for each individual. Accordingly, construction of a large amount of clinical data pertaining to more than thousands to tens of thousands of people is unfeasible from the aspect of efficiency.

Accordingly, in order to efficiently accumulate a large amount of clinical trial data for VR sickness prediction, it is required to decentralize clinical trial infrastructure, to simultaneously conduct VR sickness evaluation across remote sites, and to process data in a central server.

### Documents of Related Art

(Patent Document 1) Korean Patent No. 10-1987225, registered on Jun. 3, 2019 and titled “Apparatus and method for detecting VR sickness”.

### SUMMARY OF THE INVENTION

An object of the present invention is to extract clinical data on the degree of VR sickness of a user based on a cloud.

Another object of the present invention is to predict the degree of VR sickness based on clinical data on the degree of VR sickness based on a cloud.

A further object of the present invention is to improve the accuracy of prediction of the degree of VR sickness by generating a machine-learning model using clinical data on the degree of VR sickness.

Yet another object of the present invention is to categorize clinical data on the degree of VR sickness and to predict the degree of VR sickness for each individual or category.

Still another object of the present invention is to provide an apparatus and method for predicting and visualizing the degree of motion sickness due to image content provided in a virtual-reality service.

Still another object of the present invention is to provide an apparatus and method for predicting, online or offline, the degree of motion sickness due to various types of VR image content having no limitation as to the type of a display and for displaying the predicted degree to a user.

The objects of the present invention are not limited to the above objects, and other objects that are not mentioned will be derived from the following description.

In order to accomplish the above objects, an apparatus for a clinical trial for predicting the degree of VR sickness according to an embodiment of the present invention includes one or more processors and executable memory for storing at least one program executed by the one or more processors. The at least one program provides VR content to a user, extracts clinical data for predicting the degree of motion sickness for respective users, and transmits the clinical data to a cloud server.

Here, the clinical data may include at least one of view data based on the VR content, bio-signal data of the user, and subjective motion sickness evaluation data of the user.

Here, the view data may include at least one of image complexity of the VR content, a depth map thereof, head-tracking information of the user, and eye-tracking information of the user.

Here, the bio-signal data may be generated in the form of a feature vector by extracting at least one of a brainwave, an electrocardiogram, and a skin conductance of the user on a time axis using a sensor.

Here, the at least one program may provide a subjective motion sickness evaluation menu to the user and receive information about a selection by the user, and the subjective motion sickness evaluation data may include the information about the selection by the user.

Here, the at least one program may transmit the clinical data including a unique identifier of the user to the cloud server.

Also, in order to accomplish the above objects, a cloud server for predicting the degree of VR sickness according to an embodiment of the present invention includes one or more processors and executable memory for storing at least one program executed by the one or more processors. The at least one program receives clinical data, including at least one of view data corresponding to VR content, bio-signal data of a user, and subjective motion sickness evaluation data of the user, from a clinical trial apparatus, constructs a database by categorizing the clinical data, and analyzes the degree of VR sickness based on the clinical data.

Here, the at least one program may analyze the degree of VR sickness using a machine-learning model by receiving the clinical data as input.

Here, the at least one program may extract features data by performing preprocessing using the clinical data as input and generate the machine-learning model by performing machine learning based on the features data.

Here, the machine learning may be performed separately for a training step and a test step.

Here, the preprocessing may be configured to extract the features data based on complexity or a power spectrum after extracting the complexity through wavelet transform of the VR content included in the view data or extracting the power spectrum by performing Fast Fourier Transform (FFT) on the bio-signal data of the user.

Here, the at least one program may quantify the analyzed degree of VR sickness and transmit the quantified degree of VR sickness to the clinical trial apparatus from which the clinical data is received.

Also, in order to accomplish the above objects, a method for a clinical trial for predicting the degree of VR sickness in a cloud server according to an embodiment of the present invention includes receiving clinical data pertaining to multiple users from one or more clinical trial apparatuses; categorizing the clinical data and constructing a database; and analyzing the degree of VR sickness based on the clinical data.

Here, the clinical data may include at least one of view data based on VR content, bio-signal data of the users, and subjective motion sickness evaluation data of the users.

Here, analyzing the degree of VR sickness may be configured to analyze the degree of VR sickness using a machine-learning model by receiving the clinical data as input.

Also, the method may further include extracting features data by performing preprocessing using the clinical data as input; and generating the machine-learning model by performing machine learning based on the features data.

Here, the machine learning may be performed separately for a training step and a test step. Here, the preprocessing may be configured to extract the features data based on complexity or a power spectrum after extracting the complexity through wavelet transform of VR content included in the view data or extracting the power spectrum by performing Fast Fourier Transform (FFT) on the bio-signal data of the user.

Also, the method may further include quantifying the analyzed degree of VR sickness; and transmitting the quantified degree of VR sickness to the clinical trial apparatus from which the clinical data is received.

Also, according to the present invention, there may be provided an apparatus for predicting and visualizing the degree of fatigue caused by viewing VR content. The apparatus may include an HMD connection unit for acquiring first virtual-reality (VR) content provided in an online state; an image sequence file connection unit for acquiring second VR content provided in an offline state; a user input unit for acquiring user input; a VR sickness prediction unit for receiving VR content from at least one of the HMD connection unit and the image sequence file connection unit and analyzing the degree of fatigue caused by viewing the received VR content based on the acquired user input; and a display unit for displaying the analysis result.

The analysis may be performed using a prediction model for predicting the degree of fatigue caused by viewing VR content, the prediction model being trained in advance through machine learning.

The VR content provided in the online state may be VR content provided in an HMD device worn by a user, and the VR content provided in the offline state may be previously produced VR content.

The HMD connection unit may perform at least one function among capturing the first VR content, storing the same, displaying the same, and providing an interface for connection with the HMD device.

The image sequence file connection unit may perform at least one function among loading the second VR content, displaying the same, and managing a playlist.

The VR sickness prediction unit may perform at least one function among image preprocessing for the received VR content, calculating feature points, and deriving the degree of VR sickness based on machine learning.

The user input unit may receive user input for an item for at least one of a VR content mode, whether to automatically perform VR sickness prediction, a VR content path, and whether to play VR content.

The display unit may include at least one of an image visualization unit, a motion sickness degree visualization unit, and a program control unit. The program control unit may display a user input window for at least one of the VR content mode, whether to automatically perform VR sickness prediction, the VR content path, and whether to play the VR content in a predetermined area.

The program control unit may further display the operating state of the VR sickness prediction unit in a predetermined area.

The image visualization unit may display at least one of information about the VR content mode and VR content play information in a predetermined area.

Also, according to the present invention, there may be provided a method for predicting and visualizing the degree of fatigue caused by viewing VR content. The method may include acquiring virtual-reality (VR) content provided in at least one of an online state and an offline state; displaying the acquired VR content on a display unit; acquiring user input; analyzing the degree of fatigue caused by viewing the acquired VR content based on the acquired user input; and displaying the analysis result on the display unit.

The analysis may be performed using a prediction model for predicting the degree of fatigue caused by viewing VR, the prediction model being trained in advance through machine learning.

The VR content provided in the online state may be first VR content provided in an HMD device worn by a user, and the VR content provided in the offline state may be previously produced second VR content.

Acquiring the VR content may include performing at least one function among capturing the first VR content, storing the same, displaying the same, and providing an interface for connection with the HMD device.

Acquiring the VR content may include performing at least one function among loading the second VR content, displaying the same, and managing a playlist.

Analyzing the degree of fatigue caused by viewing the received VR content may include performing at least one function among image preprocessing for the received VR content, calculating feature points, and deriving the degree of VR sickness based on machine learning.

Acquiring the user input may include receiving user input for an item for at least one of a VR content mode, whether to automatically perform VR sickness prediction, a VR content path, and whether to play VR content.

Displaying the analysis result may include displaying a user input window for at least one of the VR content mode, whether to automatically perform VR sickness prediction, the VR content path, and whether to play the VR content in a predetermined area.

Displaying the analysis result may further include displaying the operating state for the analysis of the degree of fatigue in a predetermined area.

Displaying the analysis result may further include displaying at least one of information about the VR content mode and VR content play information in a predetermined area.

### BRIEF DESCRIPTION OF THE DRAWINGS

The above and other objects, features and advantages of the present invention will be more clearly understood from the following detailed description, taken in conjunction with the accompanying drawings, in which:

FIGS. 1 to 3 are block diagrams of an apparatus for a clinical trial for predicting the degree of VR sickness and a cloud server according to an embodiment of the present invention;

FIG. 4 is an exemplary view illustrating the use of an apparatus for a clinical trial for predicting the degree of VR sickness and a cloud server according to an embodiment of the present invention;

FIG. 5 is an exemplary view illustrating extraction of a subjective VR sickness score according to an embodiment of the present invention;

FIG. 6 is a flowchart of a method for a clinical trial for predicting the degree of VR sickness according to an embodiment of the present invention;

FIG. 7 is a view illustrating a computer system according to an embodiment of the present invention;

FIG. 8 is a block diagram illustrating the configuration of an apparatus for predicting and visualizing the degree of fatigue caused by viewing VR content according to an embodiment of the present invention;

FIG. 9 is a view illustrating a GUI provided by an apparatus for predicting and visualizing the degree of fatigue caused by viewing VR content according to an embodiment of the present invention;

FIG. 10 is a view illustrating a screen displayed in an image visualization unit in an online mode according to an embodiment of the present invention;

FIG. 11 is a view illustrating a screen displayed in an image visualization unit in an offline mode according to an embodiment of the present invention;

FIG. 12 is a view illustrating other functions provided by an image visualization unit according to an embodiment of the present invention;

FIG. 13 is a view illustrating the screen of a motion sickness degree visualization unit according to an embodiment of the present invention;

FIG. 14 is a view illustrating the screen of a program control unit according to an embodiment of the present invention; and

FIG. 15 is a view illustrating the operating state of a VR sickness prediction unit according to an embodiment of the present invention.

### DESCRIPTION OF THE PREFERRED EMBODIMENTS

The present invention will be described in detail below with reference to the accompanying drawings. Repeated descriptions and descriptions of known functions and configurations that have been deemed to unnecessarily obscure the gist of the present invention will be omitted below. The embodiments of the present invention are intended to fully describe the present invention to a person having ordinary knowledge in the art to which the present invention pertains. Accordingly, the shapes, sizes, etc. of components in the drawings may be exaggerated in order to make the description clearer.

Hereinafter, a preferred embodiment of the present invention will be described in detail with reference to the accompanying drawings.

When VR sickness clinical trial infrastructure is distributed across remote sites, unlike conventional centralized clinical trial infrastructure, an expensive bio-signal measurement device cannot be operated, VR sickness evaluation has to be autonomously conducted without the help of a guide, and it is necessary to distribute reference VR content for evaluation and an evaluation tool. Accordingly, it is required to adopt a new method.

Accordingly, the present invention intends to propose distributed VR sickness clinical trial infrastructure based on a cloud, which is capable of efficiently constructing large-scale clinical trial data for VR sickness, and this may be easily used for prediction and analysis of VR sickness caused by commercial VR content, such as a game or the like, and for development of a tool therefor.

Also, the present invention may contribute to solving a VR sickness problem in a VR content market by accurately predicting the degree of VR sickness, and may thereby extend the marketability and public availability of VR service.

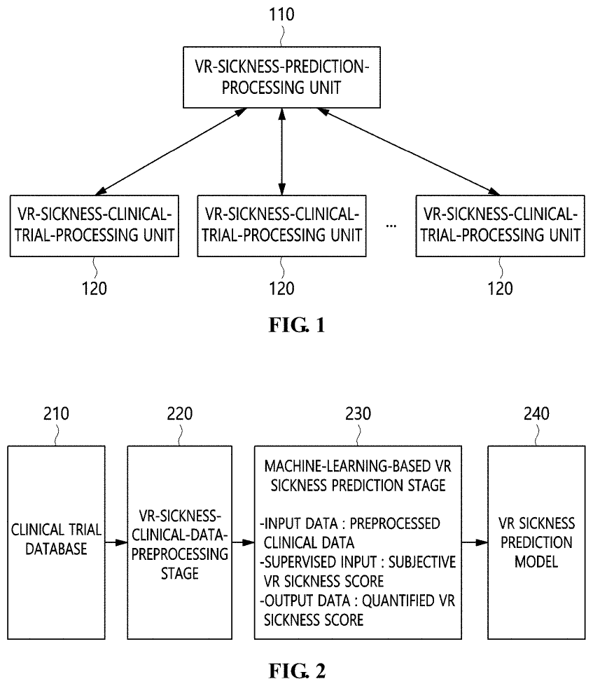

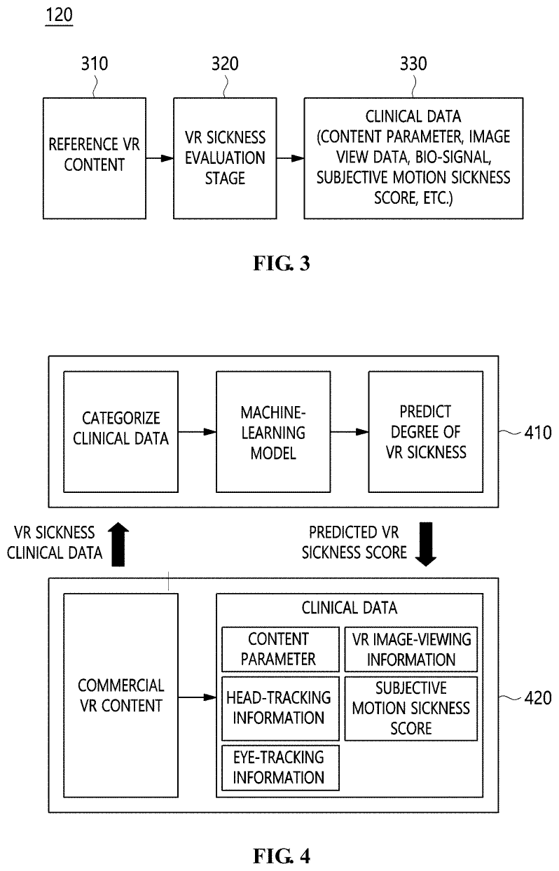

FIGS. 1 to 3 are block diagrams of an apparatus for a clinical trial for predicting the degree of VR sickness and a cloud server according to an embodiment of the present invention.

Referring to FIGS. 1 to 3, an embodiment of the present invention includes a VR-sickness-clinical-trial-processing unit 120 and a VR-sickness-prediction-processing unit 110.

The VR-sickness-clinical-trial-processing unit 120 may conduct VR sickness evaluation (320) for users using reference or commercial VR content 310 and may extract clinical data 330, and the VR-sickness-prediction-processing unit 110 may continuously accumulate the clinical data 330, received from the VR-sickness-clinical-trial-processing unit 120, in the form of a database 210 and predict the degree of VR sickness based on a machine-learning model using the clinical data.

More specifically, the VR-sickness-clinical-trial-processing unit 120 is a clinical trial station in the form of an independent client, which is distributed in a remote site, and may be the same component as an apparatus for providing a clinical trial for predicting the degree of VR sickness, which will be described later.

Here, the VR-sickness-clinical-trial-processing unit 120 may conduct a VR sickness clinical trial for each individual user using commercialized VR content or standard reference VR content 310 in order to conduct VR sickness evaluation.

Also, the VR-sickness-clinical-trial-processing unit 120 may extract clinical data 330 generated through the VR sickness clinical trial for each user and may continuously transmit the clinical data 330, which is extracted for each user, to the VR-sickness-prediction-processing unit 110 located in the cloud server.

Here, the clinical data 330 may include view data according to viewing of a VR image (content parameters, image complexity, a depth map, head-tracking information, eye-tracking information, and the like), bio-signals acquired by a patch-type sensor or a wearable sensor, subjective motion sickness evaluation scores based on survey questions answered by the subjects of the clinical trial, and the like.

Also, the VR-sickness-clinical-trial-processing unit 120 may receive the calculated degree of VR sickness from the VR-sickness-prediction-processing unit 110, check and analyze the degree of VR sickness experienced by each individual, take follow-up measures for adjusting the degree of VR sickness, or the like.

The VR-sickness-prediction-processing unit 110 may be located in the cloud server, and may be the same component as a cloud server for predicting the degree of VR sickness, which will be described later.

Here, the VR-sickness-prediction-processing unit 110 may continuously accumulate the VR sickness clinical trial data for each user, that is, the clinical data 330 received from the VR-sickness-clinical-trial-processing unit 120, in the form of a database 210, may perform preprocessing in order to apply machine learning, and may calculate the quantified degree of VR sickness by applying machine learning.

Here, the VR-sickness-prediction-processing unit may perform the process of preprocessing the clinical data using a VR-sickness-clinical-data-preprocessing stage 230.

Here, the preprocessing may be the process of extracting feature points suitable for machine learning from the original copy of clinical data for each type, and may be the process of converting view data into meaningful data by applying wavelet transform in order to extract complexity or extracting a power spectrum from bio-signals by applying Fast Fourier Transform (FFT).

The clinical data preprocessed as described above, that is, the features data, may be extracted in a data form suitable for generation of a machine-learning model 240 for predicting the degree of VR sickness.

The machine learning is executed separately for a training step and a test step for the input data, and the machine-learning model 240 may be generated after the training step.

The accuracy of prediction of VR sickness is dependent on the reliability of the machine-learning model, and as the amount of clinical model data applied to learning is greater, a highly reliable machine-learning model 240 capable of achieving enhanced accuracy may be generated.

FIG. 4 is an exemplary view illustrating the use of an apparatus for a clinical trial for predicting the degree of VR sickness and a cloud server according to an embodiment of the present invention.

Referring to FIG. 4, an arbitrary clinical trial apparatus (a cloud client 420) including a VR-sickness-clinical-trial-processing unit on a cloud may be provided with commercial VR content, and may extract clinical data based thereon.

The commercial VR content may be reference VR content for predicting the degree of VR sickness, or may be general VR content.

For example, the commercial VR content may be VR game content or a VR image that is downloadable from SteamVR, which is a commercial game platform.

Here, the arbitrary clinical trial apparatus 420 including the VR-sickness-clinical-trial-processing unit may extract VR sickness clinical data of a user (content parameters, head-tracking information, eye-tracking information, VR image-viewing information, a subjective VR sickness score, and the like) based on the VR content through a software tool.

Also, the arbitrary clinical trial apparatus 420 including the VR-sickness-clinical-trial-processing unit may deliver the VR sickness clinical data to a cloud server 410 including a VR-sickness-prediction-processing unit.

Here, the cloud server 410 including the VR-sickness-prediction-processing unit may classify the received clinical data according to need and apply machine learning, thereby predicting the degree of VR sickness.

Because the present invention enables extraction of a large amount of clinical data based on a cloud, a highly reliable machine-learning model may be generated even when bio-signals cannot be extracted due to the absence of expensive bio-signal measurement equipment, and the degree of VR sickness may be accurately predicted based only on the clinical data excluding bio-signals.

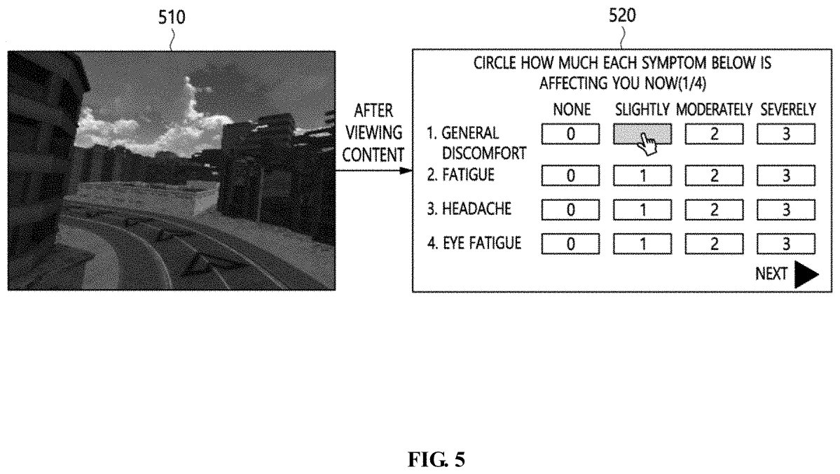

FIG. 5 is an exemplary view illustrating extraction of a subjective VR sickness score according to an embodiment of the present invention.

Referring to FIG. 5, the VR-sickness-clinical-trial-processing unit or the clinical trial apparatus for predicting the degree of VR sickness may extract a motion sickness score in such a way that a user fills out an impromptu survey online using a graphical user interface (GUI) displayed on the screen 520 after viewing a specific VR image sequence 510 in order to extract a subjective VR sickness score.

Because the user is able to control a cursor using a mouse or a VR controller in order to select a menu item on the screen 520, inconvenience that can be caused when the user is required to take off a VR HMD or to write down a motion sickness score on the survey paper offline for subjective evaluation may be avoided.

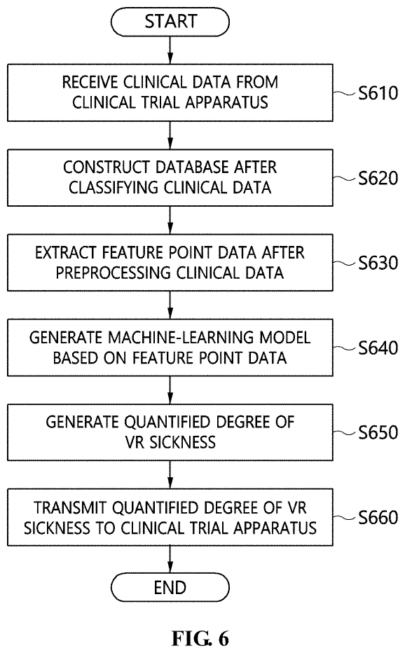

FIG. 6 is a flowchart of a method for a clinical trial for predicting the degree of VR sickness according to an embodiment of the present invention.

Referring to FIG. 6, in the method for a clinical trial for predicting the degree of VR sickness according to an embodiment of the present invention, first, a cloud server receives clinical data of multiple users from one or more clinical trial apparatuses at step S610.

Also, in the method for a clinical trial for predicting the degree of VR sickness according to an embodiment of the present invention, the clinical data is classified and stored in the form of a database at step S620.

Here, the clinical data may include at least one of view data based on VR content, bio-signal data of the users, and subjective motion sickness evaluation data of the users.

Also, in the method for a clinical trial for predicting the degree of VR sickness according to an embodiment of the present invention, features data may be extracted at step S630 by performing preprocessing using the clinical data as input.

Here, the preprocessing may be a process of extracting features data based on complexity or a power spectrum after extracting the complexity through wavelet transform of the VR content included in the view data or extracting the power spectrum by performing Fast Fourier Transform (FFT) on the bio-signal data of the user.

Also, in the method for a clinical trial for predicting the degree of VR sickness according to an embodiment of the present invention, the machine-learning model may be generated at step S640 by performing machine learning based on the features data.

Here, the machine learning may be performed separately for a training step and a test step.

Also, in the method for a clinical trial for predicting the degree of VR sickness according to an embodiment of the present invention, the degree of VR sickness is analyzed based on the clinical data.

Here, the degree of VR sickness may be analyzed using a machine-learning model by receiving the clinical data as input.

Also, in the method for a clinical trial for predicting the degree of VR sickness according to an embodiment of the present invention, the analyzed degree of VR sickness may be quantified at step S650.

Also, in the method for a clinical trial for predicting the degree of VR sickness according to an embodiment of the present invention, the quantified degree of VR sickness may be transmitted to the clinical trial apparatus from which the clinical data was received at step S660.

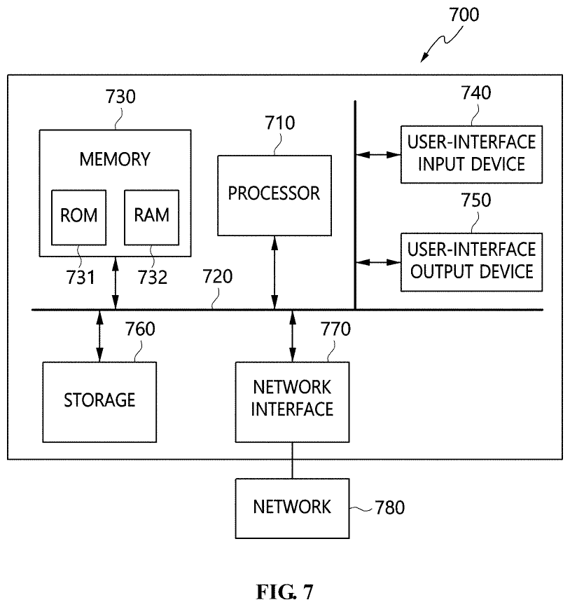

FIG. 7 is a view illustrating a computer system according to an embodiment of the present invention.

Referring to FIG. 7, an embodiment of the present invention may be implemented in a computer system including a computer-readable recording medium. As illustrated in FIG. 7, the computer system 700 may include one or more processors 710, memory 730, a user-interface input device 740, a user-interface output device 750, and storage 760, which communicate with each other via a bus 720. Also, the computer system 700 may further include a network interface 770 connected to a network 780. The processor 710 may be a central processing unit or a semiconductor device for executing processing instructions stored in the memory 730 or the storage 760. The memory 730 and the storage 760 may be any of various types of volatile or nonvolatile storage media. For example, the memory may include ROM 731 or RAM 732.

Accordingly, an embodiment of the present invention may be implemented as a nonvolatile computer-readable storage medium in which methods implemented using a computer or instructions executable in a computer are recorded. When the computer-readable instructions are executed by a processor, the computer-readable instructions may perform a method according to at least one aspect of the present invention.

Here, the apparatus for a clinical trial for predicting the degree of VR sickness according to an embodiment of the present invention includes one or more processors and executable memory for storing at least one program executed by the one or more processors. The at least one program provides VR content to a user, extracts clinical data for predicting the degree of motion sickness of each user, and transmits the clinical data to a cloud server.

Here, the clinical data may include at least one of view data based on the VR content, bio-signal data of the user, and subjective motion sickness evaluation data of the user.

Here, the view data may include at least one of the image complexity of the VR content, a depth map thereof, head-tracking information of the user, and eye-tracking information of the user.

Here, the bio-signal data may be generated in the form of a feature vector by extracting at least one of the brainwave, electrocardiogram, and skin conductance of the user on the time axis using a sensor.

Here, the at least one program may provide a menu for subjective motion sickness evaluation to the user and receive information about the selection by the user, and the subjective motion sickness evaluation data may include the information about the selection by the user.

Here, the at least one program may transmit the clinical data in which the unique identifier of the user is included to the cloud server.

Also, a cloud server for predicting the degree of VR sickness according to an embodiment of the present invention includes one or more processors and executable memory for storing at least one program executed by the one or more processors. The at least one program receives clinical data, including at least one of view data corresponding to VR content, bio-signal data of a user, and subjective motion sickness evaluation data of the user, from a clinical trial apparatus, constructs a database by categorizing the clinical data, and analyzes the degree of VR sickness based on the clinical data.

Here, the at least one program may analyze the degree of VR sickness using a machine-learning model by receiving the clinical data as input.

Here, the at least one program may extract features data by performing preprocessing using the clinical data as input, and may generate a machine-learning model by performing machine learning based on the features data.

Here, the machine learning may be performed separately for a training step and a test step.

Here, the preprocessing may be the process of extracting features data based on complexity or a power spectrum after extracting the complexity through wavelet transform of the VR content included in the view data or extracting the power spectrum by performing Fast Fourier Transform (FFT) on the bio-signal data of the user.

Here, the at least one program may quantify the analyzed degree of VR sickness and transmit the quantified degree of VR sickness to the clinical trial apparatus from which the clinical data was received.

The present invention applies functions of acquiring VR sickness clinical trial data and predicting VR sickness, which are conventionally processed by a specific clinical trial station on existing VR sickness clinical trial infrastructure in a centralized manner, to a cloud system.

Accordingly, the present invention enables a VR sickness clinical trial for a large number of users to be conducted using individual client stations for a clinical trial, which are distributed in a cloud, regardless of whether a specific clinical trial device is installed or the location thereof, whereby large-scale clinical trial data may be extracted.

Also, the present invention discloses a method enabling the degree of VR sickness to be accurately predicted by collecting and accumulating a large amount of VR sickness clinical trial result data in a cloud server.

A conventional VR sickness clinical trial requires subjects of a clinical trial to move to a specific place in order to use VR sickness clinical trial equipment and a software tool for extracting clinical data.

Also, the conventional centralized computer server predicts the degree of VR sickness by extracting clinical data after conducting a clinical trial for each user for a long period of time and by applying machine learning after a sufficiently large amount of clinical data is accumulated.

The above centralized method for a clinical trial and extraction and processing of clinical data has difficulty efficiently acquiring clinical data pertaining to a large number of subjects due to problems of clinical data accumulation, availability of a clinical trial, test place accessibility, and the like.

The conventional centralized processing method is required to greatly increase the number of subjects of a clinical trial in order to enhance the accuracy of VR sickness prediction, but is impractical due to high expenses and a long working time.

Also, in the conventional centralized processing method, it is impossible to frequently share the predicted VR sickness scores, which acts as an obstacle to research and development of VR sickness prediction technology, which can be achieved by sharing VR sickness data and predicted scores.

The present invention has advantages in that a VR sickness clinical trial may be conducted regardless of the place (at home, in schools, hospitals, companies, and the like) or whether clinical trial equipment is installed, in that clinical data can be frequently acquired, and in that the degree of VR sickness can be quickly and accurately predicted using a large amount of generalized clinical data accumulated from various regions.

Also, in order to realize highly reliable VR sickness prediction, it is essential to construct a large-scale clinical database, and the present invention facilitates construction of such a large-scale clinical database in practical terms by enabling access to VR sickness clinical trial infrastructure. Also, a cloud server may predict the degree of VR sickness based on machine learning using clinical data that is accumulated in real time by continuously receiving clinical data from individual clinical trial stations.

Accordingly, the present invention has an advantage in that it is possible to continuously improve the accuracy of prediction of VR sickness in real time.

Also, the present invention may classify large-scale clinical data, which is collected in real time, into groups (according to sex, age, occupation, or the like) for a specific purpose, and may use the same in order to analyze sensitivity to VR sickness for each individual or each group.

VR sickness cannot be uniformly handled due to a large difference in sensitivity of individuals thereto, and it is necessary to analyze the same for each group classified by sex, age, occupation, or the like. Also, the method for analysis for each group may have positive effects on alleviation of VR sickness of respective individuals or groups.

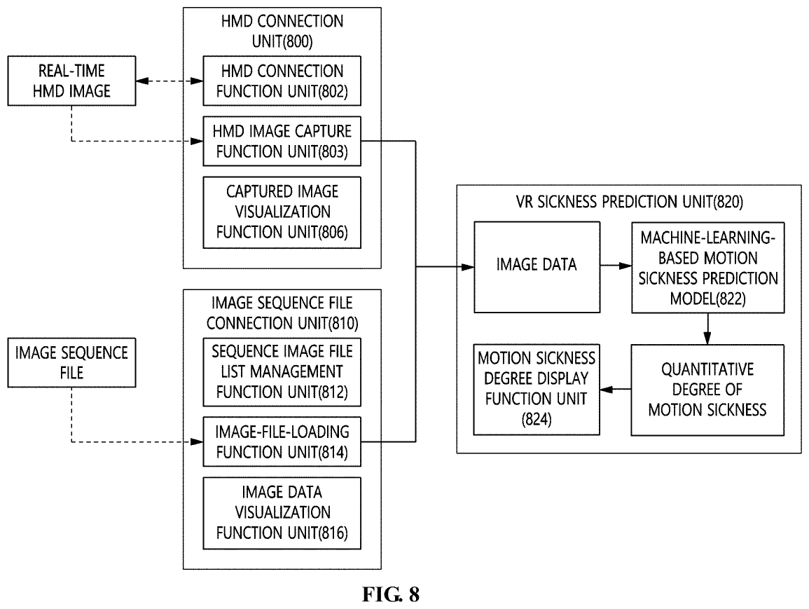

FIG. 8 is a block diagram illustrating the configuration of an apparatus for predicting and visualizing the degree of fatigue caused by viewing VR content according to an embodiment of the present invention.

Referring to FIG. 8, the apparatus for predicting and visualizing the degree of fatigue caused by viewing VR content may include an HMD connection unit 800, an image sequence file connection unit 810, and/or a VR sickness prediction unit 820. However, this illustrates only some components required for explaining the present embodiment, and the components included in the apparatus for predicting and visualizing the degree of fatigue caused by viewing VR content are not limited to the above-mentioned example.

The apparatus for predicting and visualizing the degree of fatigue caused by viewing VR content may acquire VR content in order to predict motion sickness for an online mode. Also, the HMD connection unit 810 may perform this operation.

The motion sickness prediction for the online mode (that is, an online motion sickness prediction mode) indicates the mode for visualizing the degree of VR sickness predicted when a user is viewing VR content while actually wearing an HMD, and the HMD connection unit 800 may include components for capturing and analyzing an image displayed in the HMD in real time. For example, the HMD connection unit 800 may include an HMD connection function unit 802, an HMD image capture function unit 804, and/or a captured image visualization function unit 806.

The HMD connection function unit 802 may control an interface between devices for connection with an HMD, set a capture time, and the like. Also, the HMD connection function unit 802 may support options such as image capture immediately when connection with an HMD is established, image capture after clicking a start button, and the like.

The HMD image capture function unit 804 may capture rendered images displayed on the HMD, store the same as a sequence of images, or deliver the stored images to the VR sickness prediction unit 820 after termination of image capture.

The captured image visualization function unit 806 may visualize a GUI for the captured image acquired from the HMD.

The apparatus for predicting and visualizing the degree of fatigue caused by viewing VR content may acquire VR content in order to predict motion sickness for an offline mode. Also, the image sequence file connection unit 810 may perform this operation.

The motion sickness prediction for the offline mode (that is, an offline motion sickness prediction mode) may indicate the mode for visualizing the degree of VR sickness that is predicted without an HMD by receiving previously produced VR content (e.g., recorded VR content, VR content for projection or a large display device, and the like), and the image sequence file connection unit 810 may include components for controlling and managing an image sequence for visualization of the degree of motion sickness. For example, the image sequence file connection unit 810 may include a sequence image file list management function unit 812, an image-file-loading function unit 814, and/or an image data visualization function unit 816.

The sequence image file list management function unit 812 may manage a video image playlist (e.g., a play sequence).

The image-file-loading function unit 814 may load a video image file and acquire various kinds of data therefrom.

The image data visualization function unit 816 may visualize a GUI for the image data.

The apparatus for predicting and visualizing the degree of fatigue caused by viewing VR content may predict the quantitative degree of motion sickness attributable to image data and visualize the same. Also, the VR sickness prediction unit 820 may perform this operation.

For example, the VR sickness prediction unit 820 may calculate feature points, regardless of whether it is in the online mode or offline mode, by receiving image data (that is, VR content). Also, the VR sickness prediction unit 820 may input the feature points to a motion sickness degree prediction model, which is trained in advance through machine learning, thereby quantitative predicting and outputting the degree of motion sickness. Also, the VR sickness prediction unit 820 may illustrate the degree of motion sickness and provide the same to a user. The VR sickness prediction unit 820 may include an image-preprocessing function unit (not illustrated), a feature point calculation function unit (not illustrated), a machine-learning-based motion sickness prediction model unit 822, and/or a motion sickness degree display function unit 824.

The image-preprocessing function unit may perform various kinds of preprocessing, including adjustment of the size of an input image, cropping the image, and the like.

The feature point calculation function unit may calculate information, such as a motion vector, complexity of an image on the screen, a depth thereof, and the like, using the image data. For example, the feature point calculation function unit may mathematically calculate feature points that are highly related to VR sickness.

The machine-learning-based motion sickness prediction model unit 822 may derive a quantitative motion sickness level using parameters acquired by learning the input feature points in advance. For example, the parameters may be derived by learning the relationships between the feature points and the degree of motion sickness based on clinical data pertaining to 200 or more users.

The motion sickness degree display function unit 824 may display the degree of motion sickness using a graph, and may display a representative value.

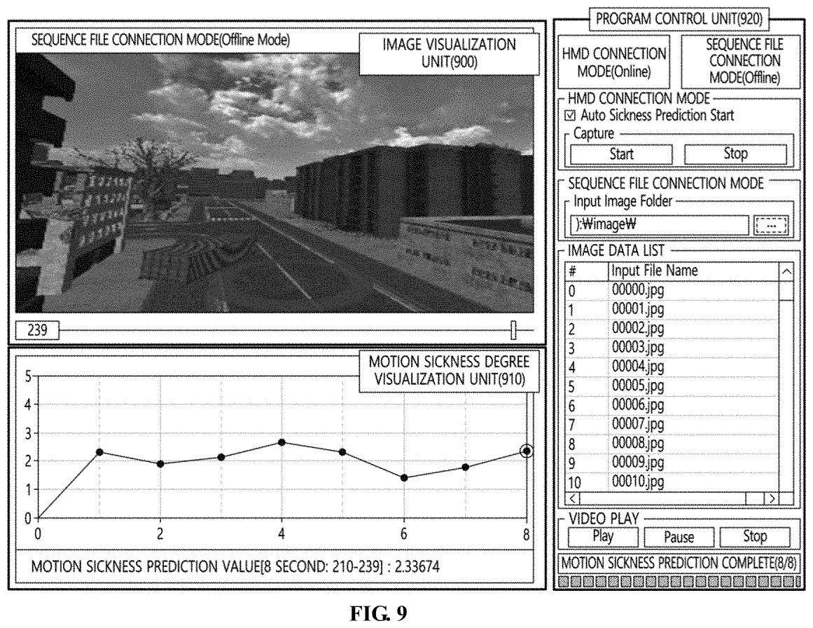

FIG. 9 is a view illustrating a GUI provided by an apparatus for predicting and visualizing the degree of fatigue caused by viewing VR content according to an embodiment of the present invention.

Referring to FIG. 9, the GUI provided by the apparatus for predicting and visualizing the degree of fatigue caused by viewing VR content may include an image visualization unit 900, a motion sickness degree visualization unit 910, and/or a program control unit 920. However, this illustrates only some components required for explaining the present embodiment, and the components included in the GUI provided by the apparatus for predicting and visualizing the degree of fatigue caused by viewing VR content are not limited to the above-mentioned example.

The image visualization unit 900 may display image data, selected from an image data list, on a display unit. Also, the image visualization unit 900 may display the current image information in a predetermined area on the display unit. The image information may include a file name, a play time, resolution, and the like. Also, the image visualization unit 900 may illustrate the image adjusted to a rendering size.

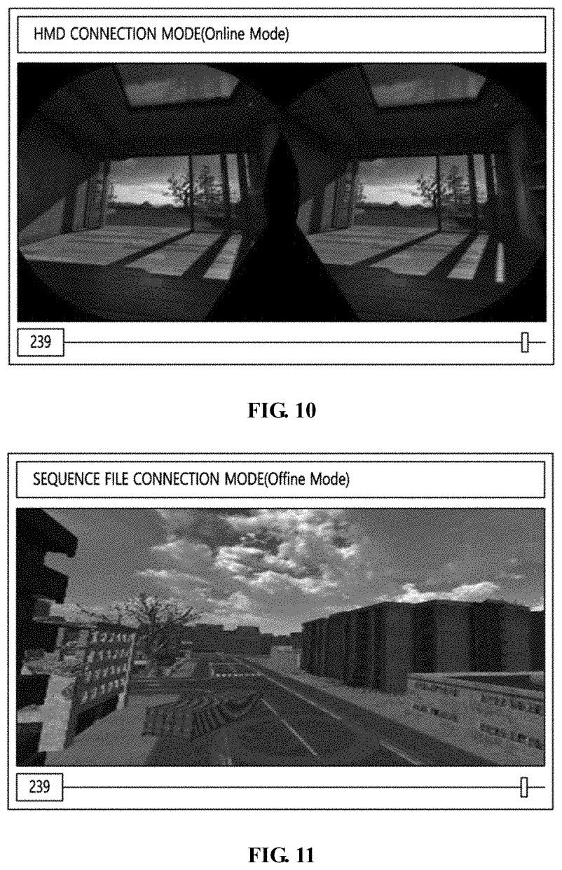

Meanwhile, in the online mode, the image visualization unit 900 may capture the stereo images rendered in the HMD actually worn by a user and display the same. FIG. 10 is a view illustrating the screen displayed in the image visualization unit in the online mode according to an embodiment of the present invention.

Also, in the offline mode, the image visualization unit 900 may visualize a corresponding image sequence file without change. FIG. 11 is a view illustrating the screen displayed in the image visualization unit in the offline mode according to an embodiment of the present invention.

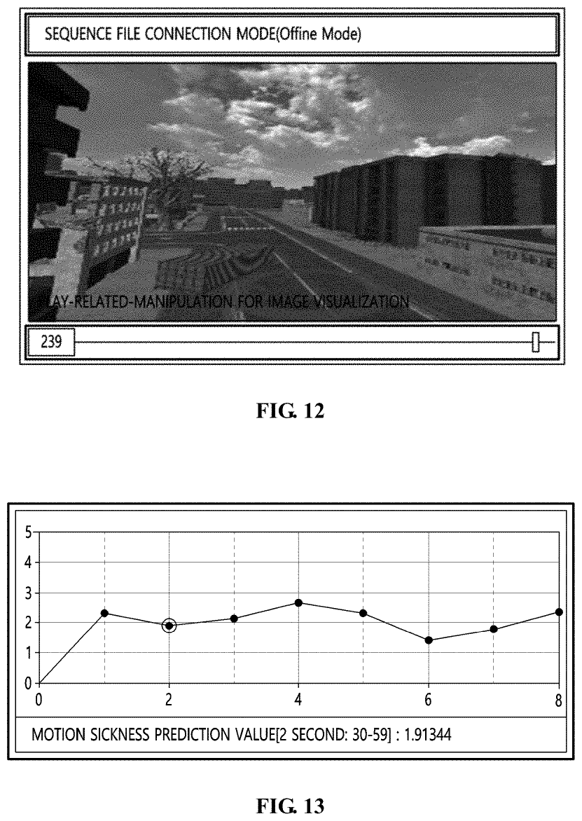

Also, the image visualization unit 900 may display mode state information in a predetermined area on the display unit. Also, the image visualization unit 900 may include an image-visualization-play-related manipulation unit. The image-visualization-play-related manipulation unit may display a frame number, the progress of a sequence, and the like in a predetermined area on the display unit, and may enable a change in a frame number in order to move to the frame desired by a user or enable dragging and moving a predetermined object indicating the progress of a sequence. FIG. 12 is a view illustrating other functions provided by the image visualization unit according to an embodiment of the present invention. Referring to FIG. 12, the mode state information of the VR image is displayed on the upper side of the display unit, and the frame number of the VR image that is currently being displayed on the display unit and the progress of a sequence are displayed on the lower side of the display unit.

The motion sickness degree visualization unit 910 may display the degree of VR sickness due to VR content as a quantitative value in a predetermined area on the display unit using a previously trained VR sickness model.

FIG. 13 is a view illustrating the screen of a motion sickness degree visualization unit according to an embodiment of the present invention.

Referring to FIG. 13, the horizontal axis may indicate time (in units of seconds), and the vertical axis may indicate the degree of motion sickness represented as a real number ranging from 0 to 5. The range displayed on the graph may be adaptively changed depending on the length of an image sequence. Also, when a cursor comes close to a point on the graph, the predicted degree of VR sickness may be specifically represented. That is, the number of frames, or a value indicating the degree of VR sickness, which is represented down to five decimal places, may be provided. For example, referring to FIG. 13, the degree of VR sickness at a time of 2 seconds (e.g., from 30th to 59th frames) may be 1.91344.

The program control unit 920 may display mode state information in a predetermined area on the display unit.

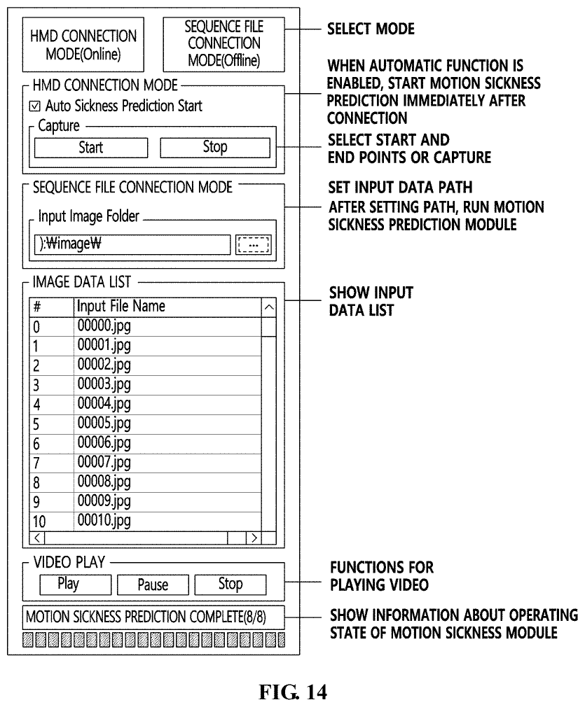

FIG. 14 is a view illustrating the screen of a program control unit according to an embodiment of the present invention.

Referring to FIG. 14, the program control unit may display a function of enabling a user to select an HMD connection mode (namely, an online mode) or a sequence file connection mode (namely, an offline mode), that is, to select a VR content mode, which indicates whether the mode is an online mode or an offline mode, in a predetermined area on the display unit. Also, in order to enable the user to select whether or not to automatically perform motion sickness prediction in the HMD connection mode, the program control unit may display the corresponding function in a predetermined area on the display unit. Here, when automatically performing VR sickness prediction is not selected, a function for setting a desired section using start and stop buttons in order to predict VR sickness caused only by the corresponding section may be displayed in a predetermined area on the display unit. When VR sickness prediction is automatically performed, a VR sickness prediction operation may be performed immediately after connection with an HMD is established.

Also, in order to enable a user to set the path of input data (that is, an image sequence) in the offline mode, the program control unit may display the corresponding function in a predetermined area on the display unit. Here, the degree of VR sickness due to the corresponding sequence section may be predicted. Also, the program control unit may display an image sequence list in a predetermined area on the display unit such that a user is able to delete/modify the image sequence list using a GUI.

Also, in order to control the video displayed in the image visualization unit using a play button, a pause button, a stop button, and the like, the program control unit may display the corresponding function in a predetermined area on the display unit.

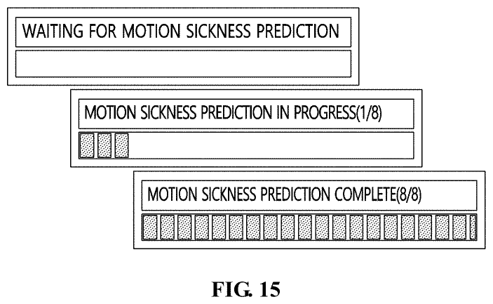

Also, the program control unit may display the operating state of the VR sickness prediction unit of FIG. 8 in a predetermined area on the display unit. FIG. 15 is a view illustrating the operating state of the VR sickness prediction unit according to an embodiment of the present invention. Referring to FIG. 15, ‘waiting for VR sickness prediction’ indicates a standby state, ‘VR sickness prediction in progress’ indicates that the VR sickness prediction operation is being performed, and ‘VR sickness prediction complete’ may indicate that the VR sickness prediction operation has been completed.

According to the present invention, an apparatus and method for predicting and visualizing the degree of motion sickness due to image content provided in a VR service may be provided.

Also, according to the present invention, an apparatus and method for predicting, online or offline, the degree of motion sickness due to various types of VR image content, which are not limited as to the type of a display, and showing the predicted degree to a user may be provided.

Also, according to the present invention, a program configured to receive image content for various kinds of VR services, to automatically predict the degree of motion sickness to be experienced by a viewer, and to display the same on a screen may be provided. The program may include the function of predicting motion sickness online, the function of predicting motion sickness offline, a motion sickness prediction module based on machine learning, the function of visualizing a motion sickness level, and various kinds of graphical user interfaces (GUI) for a user.

Also, according to the present invention, an apparatus and method for a standalone form supporting a GUI for user convenience, a form supporting both an online mode and an offline mode, or a form for visualizing a VR sickness level predicted through image analysis may be provided for all kinds of VR image content for distribution, which are not limited as to a display type.

Also, according to the present invention, there may be provided an apparatus and method capable of supporting all of a mode in which a viewer actually experiences a VR image while wearing an HMD (that is, an online mode) and a mode in which a VR sickness level is checked by receiving a recorded image/content file, rather than viewing a VR image using an HMD or a projection- or cave-type display (that is, an offline mode).

Also, according to the present invention, it may be possible to predict VR sickness due to all types of VR content, without limitation to a specific VR content production engine or platform, and to visualize the prediction result.

Also, according to the present invention, it may be possible to predict motion sickness and visualize the result thereof using only a content execution file and an image file, without a VR content project.

Also, the present invention may be used as an international standard program with regard to the production and use of VR content.

According to the present invention, clinical data on the degree of VR sickness of a user may be extracted based on a cloud.

Also, according to the present invention, the degree of VR sickness may be predicted based on clinical data on the degree of VR sickness based on a cloud.

Also, according to the present invention, a machine-learning model may be generated using clinical data on the degree of VR sickness, whereby the accuracy of prediction of the degree of VR sickness may be improved.

Also, according to the present invention, clinical data on the degree of VR sickness may be classified, whereby the degree of VR sickness for respective individuals or categories may be predicted.

Also, according to the present invention, an apparatus and method for predicting and visualizing the degree of motion sickness due to image content provided in a virtual-reality service may be provided.

Also, according to the present invention, an apparatus and method for predicting, online or offline, the degree of motion sickness due to various types of VR image content having no limitation as to the type of a display and for showing the predicted degree to a user may be provided.

Also, according to the present invention, an apparatus and method for receiving image content for various types of VR services, automatically predicting the degree of motion sickness to be experienced by a user, and displaying the same on a screen may be provided.

The effects of the present embodiments are not limited to the above-mentioned effects, and other effects that are not mentioned will be readily understood by a person of ordinary skill in the art from the accompanying claims.

As described above, the apparatus and method for a clinical trial for predicting the degree of VR sickness based on a cloud and the cloud server according to the present invention are not limitedly applied to the configurations and operations of the above-described embodiments, but all or some of the embodiments may be selectively combined and configured, so the embodiments may be modified in various ways.

What is claimed is:

1. An apparatus for a clinical trial for predicting a degree of VR sickness, comprising:
one or more processors; and
executable memory for storing at least one program executed by the one or more processors,
wherein the at least one program provides VR content to a user, extracts clinical data for predicting a degree of motion sickness for respective users, and transmits the clinical data to a cloud server.

2. The apparatus of claim 1, wherein the clinical data includes at least one of view data based on the VR content, bio-signal data of the user, and subjective motion sickness evaluation data of the user.

3. The apparatus of claim 2, wherein the view data includes at least one of image complexity of the VR content, a depth map thereof, head-tracking information of the user, and eye-tracking information of the user.

4. The apparatus of claim 2, wherein the bio-signal data is generated in a form of a feature vector by extracting at least one of a brainwave, an electrocardiogram, and a skin conductance of the user on a time axis using a sensor.

5. The apparatus of claim 2, wherein:
the at least one program provides a subjective motion sickness evaluation menu to the user and receives information about a selection by the user, and
the subjective motion sickness evaluation data includes the information about the selection by the user.

6. The apparatus of claim 1, wherein the at least one program transmits the clinical data including a unique identifier of the user to the cloud server.

7. A cloud server for predicting a degree of VR sickness, comprising:
one or more processors; and
executable memory for storing at least one program executed by the one or more processors,
wherein the at least one program receives clinical data, including at least one of view data corresponding to VR content, bio-signal data of a user, and subjective motion sickness evaluation data of the user, from a clinical trial apparatus, constructs a database by categorizing the clinical data, and analyzes the degree of VR sickness based on the clinical data.

8. The cloud server of claim 7, wherein the at least one program analyzes the degree of VR sickness using a machine-learning model by receiving the clinical data as input.

9. The cloud server of claim 8, wherein the at least one program extracts features data by performing preprocessing using the clinical data as input and generates the machine-learning model by performing machine learning based on the features data.

10. The cloud server of claim 9, wherein the machine learning is performed separately for a training step and a test step.

11. The cloud server of claim 9, wherein the preprocessing is configured to extract the features data based on complexity or a power spectrum after extracting the complexity through wavelet transform of the VR content included in the view data or extracting the power spectrum by performing Fast Fourier Transform (FFT) on the bio-signal data of the user.

12. The cloud server of claim 7, wherein the at least one program quantifies the analyzed degree of VR sickness and transmits the quantified degree of VR sickness to the clinical trial apparatus from which the clinical data is received.

13. A method for a clinical trial for predicting a degree of VR sickness in a cloud server, comprising:
receiving clinical data pertaining to multiple users from one or more clinical trial apparatuses;
categorizing the clinical data and constructing a database; and
analyzing the degree of VR sickness based on the clinical data.

14. The method of claim 13, wherein the clinical data includes at least one of view data based on VR content, bio-signal data of the users, and subjective motion sickness evaluation data of the users.

15. The method of claim 14, wherein analyzing the degree of VR sickness is configured to analyze the degree of VR sickness using a machine-learning model by receiving the clinical data as input.

16. The method of claim 15, further comprising:
extracting features data by performing preprocessing using the clinical data as input; and
generating the machine-learning model by performing machine learning based on the features data.

17. The method of claim 16, wherein the machine learning is performed separately for a training step and a test step.

18. The method of claim 16, wherein the preprocessing is configured to extract the features data based on complexity or a power spectrum after extracting the complexity through wavelet transform of VR content included in the view data or extracting the power spectrum by performing Fast Fourier Transform (FFT) on the bio-signal data of the user.

19. The method of claim 13, further comprising:
quantifying the analyzed degree of VR sickness; and
transmitting the quantified degree of VR sickness to the clinical trial apparatus from which the clinical data is received.
